(19) United States
(12) Patent Application Publication
(10) Pub. No.: US 2019/0010467 A1
(43) Pub. Date: Jan. 10, 2019

(54) METHOD FOR PREPARING CULTURED CELLS OR TISSUES FOR TRANSPLANTATION

(71) Applicant: KYOTO UNIVERSITY, Kyoto-shi (JP)

(72) Inventors: Hiroshi KAWAMOTO, Kyoto-shi (JP); Hiroshi ICHISE, Kyoto-shi (JP); Kyoko MASUDA, Kyoto-shi (JP)

(21) Appl. No.: 16/130,528

(22) Filed: Sep. 13, 2018

Related U.S. Application Data
(63) Continuation-in-part of application No. PCT/JP2017/003492, filed on Jan. 31, 2017.

(30) Foreign Application Priority Data
Mar. 16, 2016 (JP) 2016-053042

(51) Int. Cl. C12N 5/074; C12N 15/867; C12N 15/861

(52) U.S. Cl. CPC C12N 5/0696; C12N 15/867; C12N 2506/45; C12N 2502/1114; C12N 15/861

(57) ABSTRACT

Provided is a method for preparing cultured cells or tissues for transplantation, comprising at least one of the following steps 1) and 2):

1) when the cultured cells or tissues do not express an HLA-C molecule of at least one HLA-C groups expressed in the receipient's HLA-C locus, forcing the expression of an HLA-C molecule of said HLA-C group in the cultured cells or tissues, or

2) when the cultured cells or tissues are negative or weakly positive for HLA-Bw4 while the recipient is positive for HLA-Bw4, forcing the expression of an HLA molecule of HLA-Bw4 group in the cultured cells or tissues.

### CROSS REFERENCES TO THE RELATED APPLICATIONS

This application is a continuation-in-part application of international Application No. PCT/JP2017/003492, filed Jan. 31, 2017, which claims the benefit of Japanese Patent Application No. 2016-053042, filed Mar. 16, 2016. The contents of those applications are herein incorporated by reference.

### ART RELATED

The present application relates to a method for suppressing immune response in a recipient upon transplantation of cultured cells or tissues.

### BACKGROUND ART

In bone marrow transplantation, alloreactive donor's NK cells mediate antitumor activity (Blood. 110(1):433-40, 2007, the contents of the document are herein incorporated by reference). On the other hand, it had not been known whether the recipient's NK cells are involved in the rejection. Alloreactive recipient's NK cells have been reported to be involved in the rejection of the transplanted tissues (Am. J. Transplant. 11 (9):1959-64, 2011 and Transplantation. 95 (8):1037-44, 2013, the contents of the documents are herein incorporated by reference). Those two papers suggest that the alloreactive NK cells reject the transplanted tissues. It has also been reported that the reaction of the NK cells are restricted by the HLA class I molecule of the host (J. Immunol. 179(9):5977-89, 2007, the contents of the document are herein incorporated by reference). This paper uses cell lines transfected with the HLA-C1, HLA-C2 or HLA-Bw4 ligand molecule to determine the reactivity of the NK cells.

In the field of the regenerative therapy, iPS cells are widely used in the study for producing tissues for transplantation. Currently, iPS cells are mainly used in the allograft systems. Tissues regenerated from iPS cells of a donor who is homozygous for HLA, haplotypes (herein below, referred to as “HLA haplotype homo”) may be used for transplantation into not only a subject having the same haplotype as the donor in homo but also into a subject who is heterozygous for HLA haplotypes (herein below, referred to as “HLA haplotype hetero”) and one of the subject's HLA haplotypes match the donor's homozygous HLA haplotype. For the recipient's immune system, donor's HLAs are autologous, and theoretically, the rejection unlikely occurs.

Using this principle, the iPS cell stock project is now being strongly promoted in Japan. Under this project, a highly versatile iPS cell bank is created with HLA haplotype homo donors having HLA haplotypes that are frequently found in Japanese people in homozygous. The HLA haplotype homo iPS cells in the stock are distributed to research institutions as well as medical institutions so that the cells are widely used in regenerating therapies.

### SUMMARY OF THE INVENTION

An object of the present application is to provide a method for suppressing immune response of the recipient upon transplanting cultured cells or cultured tissues into the recipient. In particular, an object of the present application is to provide a method for suppressing immune response due to the activation of the recipient's NK cells upon transplanting cultured cells or tissues.

The present application provides a method for preparing cultured cells or tissues for transplantation, comprising at least one of the following steps 1) and 2):
1) when the cultured cells or tissues do not express an HLA-C molecule of at least one HLA-C groups expressed in the receipient' s HLA-C locus, forcing the expression of the HLA-C molecule of said HLA-C group in the cultured cells or tissues, or
2) when the cultured cells or tissues are negative or weakly positive for HLA-Bw4 while the recipient is positive for HLA-Bw4, forcing the expression of an HLA molecule of HLA-Bw4 group in the cultured cells or tissues.

Examples of the cultured cells or tissues may preferably include those induced from stem cells or progenitor cells, and especially, from pluripotent stem cells such as iPS cells.

The present application further provides iPS cells that are homozygous for at least. HLA-A, HLA-B and HLA-DR and having at least one additional HLA molecule that is not derived from the donor from whom the iPS cells were induced, and the additional HLA molecule is selected from the group of (1) or (2)
(1) an HLA-C molecule of HLA-C1 and/or HLA-C2 group, or
(2) an HLA molecule of HLA-Bw4 group.

The iPS cells are preferably used for producing cultured cells or tissue for transplantation that is compatible with the HLA-C groups and HLA-Bw4 groups expressed in the recipient.

The present application further provides cultured cells or tissues that are homozygous for at least HLA-A, HLA-B and HLA-DR and having at least one additional HLA molecule that is not derived from the donor from whom the cultured cells or tissues were obtained, and the additional HLA molecule is selected from the group of (1) or (2):
(1) an HLA molecule of HLA-C1 or HLA-C2 group, or
(2) an HLA molecule of HLA-Bw4 group.

The cultured cells or tissues are preferably used for transplanting into a recipient having HLA-C molecules of both HLA-C1 and C2 groups and/or into a recipient who is positive for HLA-Bw4.

Further more, the present application provides a method for creating an iPS cell bank for transplantation into recipients who are heterozygote for the HLA haplotypes, which comprising the steps of:
(1) Preparing iPS cells induced from a donor who is homozygous for at least HLA-A, HLA-B and HLA-DR,
(2-1) when the donor has HLA-C1/C1 ligand molecules at the HLA-C locus, introducing a gene encording an HLA-C2 ligand molecule into the iPS cells; when the donor has HLA-C2/C2 ligand. molecules at the HLA-C locus, introducing a gene encording an HLA-C1 ligand molecule into the iPS cells, and/or
(2-2) when the donor is negative or weakly positive for HLA-Bw4, introducing a gene encoding an HLA-Bw4 ligand molecule into the iPS cells,
(3) storing the iPS cells obtained in step (2-1) and/or 2) in connection with information regarding HLA of the donor and the HLA molecule introduced into the iPS cells. Cells suitable for transplanting into a given recipient can be chosen from the iPS cell bank so that the cells are compatible with the HLA-C ligand molecules in the recipient and/or the presence or absence, and the type of the HLA-Bw4 ligand molecule in the recipient.

According to the method of the present application, rejection against the transplanted cells or tissues by the NK cells of the recipient that may occur when the cultured cells or tissues to be transplanted do not express any HLA-C molecule belonging to the HLA-C group (s) expressed in the recipient may be avoided. In addition, rejection against the transplanted cells or tissues by the NK cells of the recipient that may occur when the cultured cells or tissues are negative or weakly positive for HLA-Bw4, while the recipient is positive for HLA-Bw4 may also be avoided.

For example, assuming a therapy in which iPS cells obtained from an iPS cell hank composed of cells homozygous for HLA haplotypes are trasplanted into a recipient having HLA haplotypes one of which matches the homozygous HLA haplotype of the iPS cells, 20-30% of recipients who are target for the therapy have both HLA-C1 and C2 ligand molecules. Regenerative therapies in which cells or tissues derived from an HLA haplotype homo donor are transplanted into an HLA haplotype hetero recipient are important for the development of the therapies. The method provided here can avoid the rejection reaction that may occur upon said transplantation and therefore, is very useful.

### BRIEF DESCRIPTION OF THE DRAWINGS

FIG. 1 shows NK cells obtained from a healthy volunteer hetero-1 that were sorted by the expression of KIR receptors.

FIG. 2 shows effects of each fraction of the NK cells of hetero-1 on T cells differentiated from iPS cells induced from a volunteer homo-A, T cells differentiated from iPS cells induced from homo-A which were forced to express C*04:01:01, and T cells of hetero-1 (auto T). The effects are shown as the ratio of CD107a positive cells.

FIG. 3 shows the cytotoxic effects of the hetero-1 NK cells on the cells shown in FIG. 2. The effects are shown as the ratio of Annexin V positive cells that means dead cells.

FIG. 4 shows effects of each fraction of the hetero-1 NK cells on vasuclar endotherial cells differentiated from homo-A iPS cells (homo-A), endotherial cells differentiated from homo-A iPS cells which were forced to express C*04:01:01 (homoA+C*04:01:01), and vascular endothelial cells of hetero-1 (auto). The effects are shown as the ratio of CD107a positive cells.

FIG. 5 shows effects of each fraction of the NK cells of a healthy volunteer hetero-2 on vasuclar endotherial cells differentiated from the homo-B iPS cells (homo-B), endotherial cells differentiated from homo-B iPS cells which were forced to express C*04:01:01 (homoB+HLA-C*15:02:01), and vascular endothelial cells of hetero-2 (auto). The effects are shown by the ratio of CD107a positive cells.

FIG. 6 shows NK cells obtained from a helathy volunteer Donor-NK1 that were fractionated by FACS with an antibody against KIR3DL1 that is an inhibitory receptor specific for the HLA-Bw4 ligand and an antibody against KIR2DL3 that is an inhibitory receptor specific for the HLA-C1 ligand.

FIG. 7 shows cytotoxic activity of each fraction of NK cells of Donor-NK1 monocytes of Donor-A and Donor-B as target cells. The effects are shown by CD107a positive cells.

### EMBODIMENT FOR CARRYING OUT THE INVENTION

NK cells have a Killer Immunoglobulin-like receptor (KIR) molecule that is an inhibitory receptor. This receptor KIR determine whether the tissue is autologous by the type of the HLA class I molecules, especially, the type of HLA-C molecules. That is, when the KIR recognises tissues or tumors not expressing the HLA molecules which is the ligand for the KIR, for example transplanted tissues and tumor cells not expressing HLA, the mechanism to inhibit the activation of the NK cells does not work and killer activity is exerted. When the donor's cells or tissues do not express an HLA molecule of the HLA-C group that is recognized by the KIR repertoire of the recepient, the NK cells of the recipient will become cytotoxic against the transplanted donor's cells or tissues.

Human HLA-C alleles are divided into two categories, HLA-C1 and HLA-C2 groups. KIR2DL2 and/or KIR2DL3 bind to an HLA-C molecule of HLA-C1 group (hereinafter, referred to as “an HLA-C1 ligand molecule”) and KIR2DL1 binds to an HLA-C molecule HLA-C2 group (hereinafter, referred to as “an HLA-C2 ligand molecule”). By the binding of the HLA-C molecule to the specific KIR, the activation of the NK cells will be suppressed. When an individual has HLA-C1/C1 ligand molecules, his/her NK cells express KIR2DL2 and/or KIR2DL3. The activation of NK cells against the autologous tissues is inhibited when the HLA-C1 ligand molecule on the autologous tissue binds to the inhibitory receptor When another individual has HLA-C2/C2 or HLA-C1/C2 ligand molecules, his/her NK cells express KIR2DL1 and when a HLA-C2 ligand molecule on a cell binds to the receptor, activation of the NK cells against the cell will be suppressed.

A recipient having HLA-C1/C2 ligand molecules express both KIR2DL1 and KIR2DL2/KIR2DL3 on his/her NK cells. In general, allograft of cultured cells or tissues will be conducted between HLA-matched donor and recipient. Although the degree of HLA-matching needs not to be perfect, the HLA matching between the donor and recipient is necessary to achieve a certain level. When the donor's HLA-C ligand molecules are HLA-C1/C1 or HLA-C2/C2 and the recipient has HLA-C1/C2 ligand molecules, the mechanism to inhibit the activation of NK cells in response to the HLA-C ligand not expressed in the donor's cells or tissues will not work and the recipient's NK cells will attack the transplanted cells or tissues.

Similar problem may be observed when donor is negative or weakly positive for HLA-Bw4. A part of HLA-B genotypes act as ligands for inhibitory receptors on NK cells and are called as “HLA-Bw4 ligand”. A part of HLA-A genotypes also act as an HLA-Bw4 ligand, however they stimulate the NK cell inhibitory receptor only weakly. Independent from the HLA-C ligands, cultured cells or tissues derived from a donor who is negative for or weakly positive for HLA-Bw4 may also be attacked from a recipient's NK cells when transplanted to the recipient who is positive for HLA-Bw4.

A recipient positive for HLA-Bw4 has KIR3DL1 on his/her NK cells. When cells or tissues differentiated from iPS cells induced from a donor who is negative or weakly positive for HLA-Bw4 are transplanted to the recipient, the mechanism for inhibiting NK cell activation will not work and the transplanted cells or tissues are attacked by the recipient's NK cells and rejected.

In this application, “HLA-Bw4 ligand” may include HLA molecules of B*07:36, B*08:02, B*08:03, B*15:13, B*15:16, B*15:17, B*15:23, B*15:24, B*40:13, B*40:19 and B*47:01. The “weakly positive HLA-Bw4 ligand” may include HLA molecules of A*23:01, A*24:01 and A*25:01. A cell or tissue is weakly positive for HLA-Bw4 when the cell or tissue expresses the “weakly positive HLA-Bw4 ligand” but not expresses any one of the HLA-Bw4 ligand as above. Examples of HLA-B molecules that are negative for HLA-Bw4 include B*27:08, B*27:12 and B*37:03N, B*44:09, B*44:15, B*47:02, B*47:03, B*51:50 and B*53:05.

In one embodiment of the present application, when the cultured cells or tissues do not express an HLA-C molecule of at least one HLA-C groups expressed, in the receipient's HLA-C locus, the HLA-C molecule of said HLA-C group is forced to express in the cells or tissues.

For example, when the donor has HLA-C1/C1 ligand molecules, an HLA-C2 molecule is forced to express in the cells or tissues of the donor. When the donor has HLA-C2/C2 ligand molecules, an HLA-C1 ligand molecule is forced to express in the cells or tissues of the donor. Then, thus modified cells from the donors are recognized by the NK cells of the recipient who has HLA-C1/C2 ligand molecules. That is, the HLA-C molecules on thus modified cells or tissues bind to both inhibitory receptors specific for respective HLA-C1 and HLA-C2 ligands on the NK cells of the recipient and accordingly, the rejection of the cells or tissues due to the NK cells of the recipient is avoided or attenuated.

In another embodiment of the present application, when the cultured cells or tissues are negative or weakly positive for HLA-Bw4, an HLA-Bw4 ligand molecule is forced to express in the cells or tissues. By forcing to express the HLA-Bw4 ligand molecule that is not expressed in the cultured cells or tissues, the cells or tissues can avoid or attenuate the rejection due to the NK cells of the recipient. That is, when the recipient is positive for HLA-Bw4, the HLA-Bw4 molecule that is forced to express in the cultured cells or tissues will bind to the receptor on the NK cells of the recipient specific for the HLA-Bw4 ligand and then, the rejection due to the recipient's NK cells is avoided or attenuated.

The cultured cells or tissues for transplantation used in the present application are cultured cells or tissues that are used for transplanting into a recipient. Preferably, the cultured cells or cultured tissues are those derived from stem cells or progenitor cells.

Examples of stem cells may include somatic stem cells such as neural stem cells, hematopoietic stem cells, mesenchymal stem cells and dental pulp stem cells and pluripotent stem cells. Pluripotent stem cells refer to stem cells having pluripotency, i.e. an ability to differentiate into many types of cells in the body, and self-propagation ability. Examples of pluripotent stem cells may include embryonic stem cells (ES cells), nuclear transfer embryonic stem cells (ntES cells) derived from cloned embryos, embryonic germ cells (EG cells), and induced pluripotent stem cells (iPS cells). ES cells and iPS cells are preferable and especially, iPS cells are preferably used.

Examples of progenitor cells may include tissue progenitors such as pluripotent hematopoietic progenitors, T cell progenitors, monocytes, erythroblasts, megakaryoblasts, osteoblasts, neural progenitors, and hepatic progenitors.

More preferably, the cultured cells or tissues for transplantation may be those differentiated from “haplotype homo iPS cells”. Haplotype homo iPS cells are iPS cells induced from the cells of a donor who is homozygous for HLA haplotypes.

iPS cells homozygous for HLA haplotypes used in the method of the present application may be those induced from a donor who is confirmed to be homozygous for at least three loci including HLA-A, HLA-B and HLA-DRB. Preferably, the iPS cells may be induced from a donor who is homozygous for four loci including HLA-A, HLA-B, HLA-DPB and HLA-C. Induced pluripotent stem (iPS) cells can be prepared by introducing specific reprogramming factors to somatic cells. iPS cells are somatic cell-derived artificial stem cells having properties almost equivalent to those of ES cells and the procedure for preparing iPS cells have been known to the art (K. Takahashi and, S. Yamanaka (2006) Cell, 126:663-676;K, Takahashi et al. (2007), Cell, 131:861-872; J. Yu et al. (2007), Science, 313:1917-1920; Nakagawa, N. et al., Nat. Biotechnol. 26:101-106 (2008); and WO 2007/069666).

A project for creating an iPS cell stock involving iPS cells established from cells derived from healthy volunteers with a homozygous HLA haplotype is now in progress at Faculty of Medicine, Kyoto University. iPS cells used in the present application may be obtained from the iPS cell stock.

Alternatively, iPS cells may be T-iPS cells that are induced from a T cell of a donor with a homozygous HLA haplotype. T-iPS cells that are iPS cells induced from a human T cell can be established by a known procedure, for example based on the description of WO2013/176197.

In one embodiment of the method of the present application, cultured cells or tissues for transplantation derived from a donor having ligand molecules of HLA-C1/C1 or HLA-C2/C2 are forced to express either HLA-C1 or HLA-C2 ligand molecule which the donor does not have so that the cells or tissues express both HLA-C ligands. The HLA-C1 or HLA-C2 ligand molecule to be expressed in the cultured cells or tissues may be the same or different from the HLA-C molecule of the recipient as long as the molecule belongs to the HLA-C1 or HLA-C2 group which the donor does not have. Preferably, the HLA-C molecule to be expressed in the cultured cells or tissues is the same HLA-C molecule in the recipient.

In one embodiment of the method of the present application, cultured cells or tissues for transplantation derived from a donor who is negative or weakly positive for HLA-Bw4, the cultured cells or tissues to be transplanted are forced to express a HLA-Bw4 ligand molecule. HLA-Bw4 ligand molecule may be any of those having relatively high affinity to the HLA-Bw4 specific receptor on NK cells. Preferably, the HLA-Bw4 ligand molecule to be expressed in the cultured cells or tissues may be the same as HLA-Bw4 ligand molecule expressed on the recipient's cells.

Upon inducing the differentiation of stem cells or progenitor cells derived from a donor into desired cultured cells or tissues, the original HLA molecules are maintained in general. In one embodiment of the present method, the desired HLA molecule is expressed in the differentiated cultured cells or tissues. The procedure for differentiating stem cells or progenitor cells into desired cells or tissues may be any procedures that have been known to the art.

The expression of the desired HLA-C and/or HLA-Bw4 ligand molecule in the cells or tissues differentiated from stem cells or progenitor cells may be in a manner that the inhibitory receptor on the NK cells recognize the expressed molecule. The expression may be permanent or transient. For forcing the expression of HLA-C and/or HLA-Bw4 ligand molecules in the cells or tissues, the cells or tissues may be contacted with a gene or gene product of the desired HLA-C and/or HLA-Bw4 ligand molecule.

For example, HLA-C and/or HLA-Bw4 ligand proteins are introduced into the differentiated cultured cells or tissues by sprinkling the protein to the cells, by means of lipofection, by fusion of cell-permeable peptides (e.g. HIV-derived TAT or polyarginine) and HLA-C and/or HLA-Bw4 ligand proteins or by means of microinjection.

Alternatively, a DNA encoding the desired HLA-C and/HLA-Bw4 molecule may be introduced into the cultured cells or tissues by using a vector including virus, plasmid and artificial chromosome vectors; by means of lipofection; by using liposomes; or by means of microinjection. Examples of the viral vectors include retrovirus vectors, lentivirus vectors (these are described in Cell, 126, pp. 663-676, 2006; Cell, 131, pp. 861-872, 2007; and Science, 318, pp. 1917-1920, 2007), adenovirus vectors (Science, 322, 945-949, 2008), adeno-associated virus vectors and Sendai virus vectors (WO 2010/008054). Examples of the artificial chromosome vector include human artificial chromosome (HAC), yeast artificial chromosome (YAC), and bacterial artificial chromosome (BAC and PAC). Examples of the plasmid which may be used include plasmids for mammalian cells (Science, 322:949-953, 2008). The vector may contain a regulatory sequence(s) such as a promoter, enhancer, ribosome binding sequence, terminator and/or polyadenylation site to enable expression of the transgenes; and, as required, a sequence of a selection marker such as a drug resistance gene (e.g., kanamycin-resistant gene, ampicillin-resistant gene or puromycin-resistant gene), thymidine kinase gene or diphtheria toxin gene; a gene sequence of a reporter such as the green-fluorescent protein (GFP), β-glucuronidase (GUS) or FLAG. Further, in order to remove, after introduction of the gene into the cultured cells or tissues and expression of the same, the gene encoding the HLA-C and/or HLA-Bw4 ligand molecule, or both the promoter (s) and the gene encoding the HLA-C/HLA-Bw4 molecule linked thereto, the vector may have LoxP sequences upstream and downstream of these sequences.

In the case where an RNA encoding HLA-C and/or HLA-Bw4 ligand molecule is introduced, the RNA may be introduced by means of lipofection or microinjection, and an RNA into which 5-methylcytidine and pseudouridine (TriLink Biotechnologies) were incorporated may be used in order to suppress degradation (Warren L, (2010) Cell Stem Cell. 7:618-630)

In a preferred embodiment, the cultured cells or tissues may be those differentiated from iPS cells. It has been well known that iPS cells can be differentiated into various cells and tissues. For example, iPS cells may be differentiated into various cells by procedures known for differentiating ES cells. Procedures for differentiating the ES/iPS cells into neural stem cells (JP2002-2914699A), into pancreatic stem cells (JP2004-121165A), into hematopoietic stem cells (JP2003-505006A), and differentiating through the formation of embryoid body (JP2003-523766A) may be employed to provide, for example, cardiomyocytes, blood cells, nerve cells, vascular endothelial cells, insulin secreting cells. In addition, new methods of producing various products from iPS cells such as method of producing retinal pigment epithelial cell sheet (WO2012/115244) and method for inducing immune eels (WO2016/010148, WO2016/010153, WO2016/010154, WO2016/010155) have been proposed. In the present application, any of the known methods may be employed for differentiating iPS cells into the desired cells or tissues.

The stem cells, e.g. iPS cells, may be introduced with the desired HLA-C and/or HLA-Bw4 ligand molecules and then, differentiated into the desired cells or tissues. Genes encoding HLA-C and/or HLA-Bw4 ligand molecule may be incorporated into the genome by means of lentiviral or retroviral vectors. The HLA-C and/or HLA-Bw4ligand molecules incorporated into the genome will be maintained as they in the cells differentiated from the iPS cells.

In one embodiment, the desired cells differentiated from iPS cells express an HLA-C ligand molecule in addition to and other than HLA-C1/C1 or HLA-C2/C2 ligand molecules that the original iPS cells have. The cultured cells or tissues express both HLA-C1 ligand molecule and HLA-C2 ligand molecule, and therefore, when the cells or tissues are transplanted into a recipient having HLA-C1/C2 ligands, the HLA-C1 and HLA-C2 ligand molecules bind to the inhibitory receptors on the recipient's NK cells specific for HLA-C1 and HLA-C2, respectively. Then, the activation of the recipient's NK cells is avoided.

In one embodiment, the desired cells differentiated from iPS cells express an HLA-Bw4 ligand molecule even when the cells or tissues may be those differentiated from iPS cells that do not originally have any HLA-Bw4 ligand. When the cells or tissues are transplanted into a recipient who is positive for HLA-Bw4, the HLA-Bw4 ligand molecule binds to the inhibitory receptor on the recipient's NK cells specific for HLA-Bw4 and the activation of the recipient's NK cells is avoided.

The present application further provides a method for creating an iPS cell bank for a recipient with heterozygous HLA haplotypes, comprising the steps of:

(1) providing iPS cells established from donors who are homozygous for at least HLA-A, HLA-B and HLA-DRB loci,

(2-1) introducing a gene encoding an HLA-C2 ligand molecule into the iPS cells when the HLA-C locus of the donor has HLA-C1/C1 ligand molecules, or introducing a gene encoding an HLA-C1 ligand molecule into the iPS cells when the HLA-C locus of the donor has HLA-C2/C2 ligand moleculess, and/or

(2-2) introducing a gene encoding a HLA-Bw4 ligand molecule into the iPS cells, when the donor is negative or weakly positive for HLA-Bw4, and

(3) storing the iPS cells obtained in step (2-1) and/or (2-2) in connection with information regarding HLA of each donor and the introduced HLA-C and/or HLA-Bw4 ligand molecules. In this method, the HLA-C locus of the donor is preferably homozygous.

The iPS cell bank of the present application is preferably used in connection with an iPS cell bank established from cells of donors who are homozygous for HLA haplotypes. The iPS cell bank provided herein is preferably used for preparing tissues or cells suitable for transplantation according to the HLA-C and HLA Bw4 ligand molecules that the recipient has.

That is, the iPS cell bank provided herein comprises the following (1) and/or (2) in addition to the iPS cells induced from donors who are homozygous for HLA haplotypes,

(1) iPS cells induced from donors having HLA-C1/C1 ligand molecules and introduced with a gene encoding an HLA-C2 ligand molecule, and iPS cells induced from donors having HLA-C2 /C2 ligand molecules and introduced with a gene encoding HLA-C1 ligand molecule, and/or

(2) iPS cells induced from donors who are negative or weakly positive for HLA-Bw4 and introduced with a gene encoding an HLA-Bw4 ligand molecule. The iPS cells are stored in connection with information regarding HLA of the donor and HLA-C and/or HLA-Bw4 ligand molecules induced in the iPS cells.

The present application further provide a method for suppressing activation of recipient's NK cells upon transplanting the cultured cells or tissues for transplantation which comprises administering a substance that inhibits activation of NK cells together with the cells or tissues. In the specification and claims, the “substance that inhibits activation of NK cells” may be beads immobilized with, solubilized molecule of, or tetramer of an HLA-C ligand molecule and/or HLA-Bw4 ligand molecule that is not expressed in the cultured cells or tissues and expressed in the recipient. Alternatively, the substance that inhibits activation of NK cells may be a stimulating antibody against the inhibitory receptor (KIR) specific for those ligands.

The solubilized HLA molecules may be obtained, for example, by cleavage of the transmembrane portion, fusion with the Fc portion of the antibody molecule and tetramerization. Those substances that inhibit activation of NK cells may be added to the medium used upon transplanting the cells or tissues, or administered to the recipient before or after the transplantation.

The present application further provides a method for preparing cultured cells or tissues for transplantation which comprises at least one step selected from the group consisting of the following 1) and 2):

1) when the cultured cells or tissues do not express an HLA-C molecule of at least one HLA-C groups expressed in the receipient s HLA-C locus, forcing the expression of a stimulating antibody against the inhibitory receptor of the NK cells specific for the HLA-C molecule of said HLA-C group in the cultured cells or tissues, or

2) when the cultured cells or tissues are negative or weakly positive for HLA-Bw4 while the recipient is positive for HLA-Bw4, forcing the expression of a stimulating antibody against the inhibitory receptor of the NK cells specific for the HLA molecule of HLA-Bw4 group in the cultured cells or tissues.

The present application will be explained in more detail with examples below. The examples do not limit the scope of the invention disclosed herein in any means.

### EXAMPLE 1

### 1) Preparation of the Re-Generated T Cells

iPS cells (T-iPS cells) were established from a T cell of a healthy donor (homo-A) who was homozygous for HLA haplotypes. The obtained iPS cells were differentiated into CD8 single positive T cells (re-generated T cells). Another iPS cells were established from a T cell of a healthy donor (hetero-1) who has heterozygous HLA haplotypes one of which matches the homo-A's HLA haplotype in the same manner as above. The iPS cells were differentiated into CDB single positive cell s. iPS cells were established from the T cell according to the procedures taught by WO2016/0101535. The obtained iPS cells were differentiated into CD8 single positive T cells. The haplotypes of homo-A and hetero-1 are shown in table 1 below. The HLA-C 14:03, 12:02 are HLA-C1 ligand molecules and HLA-C 04:01 and 15:02 are HLA-C2 ligand molecules. Accordingly, homo-A has HLA-C1/C1 ligand molecules and hetero-1 has HLA-C1/C2 ligand molecules.

TABLE 1

| | HLA-A | HLA-B | HLA-C | HLA-DRB1 |
|---|---|---|---|---|
| homo-A | 33:03 | 44:03 | 14:03 | 13:02 |
| | 33:03 | 44:03 | 14:03 | 13:02 |
| hetero-1 | 31:01 | 48:01 | 04:01 | 04:03 |
| | 33:03 | 44:03 | 14:03 | 13:02 |

2) Differentiation of T-iPS Cells into T Cells

Media used were as follows:

TABLE 2
Medium A: for maintenance of OP9 stromal cells

| | | |
|---|---|---|
| contents | amount added | final conc. |

| | | | |
|---|---|---|---|
| αMEM medium | 500 | ml | |
| FCS | 125 | ml | 20% |
| penicillin-streptomycin | 6.25 | mL | 1% |
| solution* | | | |
| Total | 631.25 | mL | |

*Mixture of Penicillin (10,000 U/ml) and Streptomycin (10,000 μg/ml). The final concentrations were 100 U/ml and 100 μg/ml, respectively.

TABLE 3
Medium B: for inducing differentiation of T cells

| | | |
|---|---|---|
| contents | amount added | final conc. |

| | | | |
|---|---|---|---|
| αMEM medium | 500 | mL | |
| FCS | 125 | mL | 20% |
| penicillin-streptomycin | 5 | mL | 1% |
| solution* | | | |
| hrIL-7 (stock: 10 μg/mL) | 315 | μL | 5 ng/mL |
| hrFlT-3L (stock: 10 μg/mL) | 315 | μL | 5 ng/mL |
| hrSCF (stock: 10 μg/mL) | 630 | μL | 10 ng/mL |
| Total | 631.26 | mL | |

*Mixture of Penicillin (10,000 U/ml) and Streptomycin (10,000 μg/ml). The final concentrations were 100 U/ml and 100 μg/ml, respectively.

### Preparation of OP9 Cells

Six milliliters (6 mL) of 0.1% gelatin solution in PBS was added to a 10 cm dish (Falcon) and incubated for 30 or more minutes at 37° C. OP9 stromal cells were detached from a confluent culture dish with trypsin/EDTA solution and about ¼ of the obtained cells were added to the gelatin-coated 10 cm cell culture dish. 10 mL of medium A was added to the cell culture dish.

Four days after, 10 mL of medium A was added to the dish to give final amount of 20 mL.

Induction of Hematopoietic Progenitor Cells from iPS Cells

The medium in the OP9 stromal cell culture to be used for the co-culture was aspirated and replaced with fresh medium A. The medium in the human iPS cell culture dish was also aspirated and 10 mL of fresh medium A was added there. The human iPS cell mass was cut with an EZ-passage roller. The cut iPS cell mass was suspended by means of a pipetman with a 200 μL tip. The number of the iPS cell clusters was visually counted and approximately 600 clusters were seeded on the OP9 cells.

Three or more dishes per clone of iPS cells were used, and when subculturing, the cells in all dishes were once pooled in one dish and then redistributed to the same number of dishes to reduce the disparity between the dishes.

Day 1: (the medium was replaced)

Whether or not the iPS cell mass adhered to the dish, and started to differentiate were observed. The cell culture medium was replaced with 20 mL of fresh medium A.

Day 5: (a half of the medium was replaced)

A half of the cell culture medium was replaced with 10mL of fresh medium A.

Day 9: (a half of the medium was replaced)

A half of the cell culture medium was replaced with 10 mL of fresh medium A.

Day 13 (Induced mesodermal cells were transferred from OP9 cell layer onto OP9/DLL1 cell layer)

Cell culture medium was aspirated to remove and the surface of the cultured cells were washed with HBSS (+Mg+Ca) to washout the cell culture medium. 10 mL of Collagenase IV 250U in HBSS (+Mg+Ca) solution was added to the dish and incubated for 45 minutes at 37° C.

The collagenase solution was removed by aspiration and the cells were washed with 10 mL of PBS(−). Then, 5 mL of 0.05% trypsin/EDTA solution was added to the dish and the dish was incubated for 20 minutes at 37° C. After the incubation, the sheet like cell aggregates peeled from the bottom of the dish and the cell aggregates were mechanically fragmented to smaller sizes by means of pipetting.

Thus treated cells were added with 20 mL of fresh medium. A and cultured for more 45 minutes at 37° C. The culture medium containing the floating cells was passed through a 100 μm mesh and the cells were collected. The cells were then centrifuged at 1200 rpm for 7 minutes at 4° C. The obtained pellet was suspended in 10 mL of medium B. One-tenth of the suspension was separated and used for the FACS analysis. The remaining cell suspension was seeded on new dishes containing OP9/DLL1 cells. Cell suspensions obtained from several dishes were pooled and the pooled cells were then redistributed to the same number of dishes.

In order to ascertain whether or not hematopoietic progenitor cells were contained in the obtained cells, FACS analysis was carried out using anti-CD34 antibody and anti-CD43 antibody. The results are shown in FIG. 4. A sufficient number of cells could be confirmed in the CD34^{low}CD43⁺ cell fraction, and therefore, it was confirmed that hematopoietic progenitor cells were induced.

Induction of T cells from the Hemapoietic Progenitor Cells

Then, the obtained cells were seeded on new dishes containing OP9/DLL1 cells. In this step, cell sorting for the CD34^{low}CD43⁺ cell fraction was not performed. When this fraction is sorted, the efficiency of differentiation of T cells could be reduced in comparison with the case where sorting is not performed due to the decrease of the cells or damage to the cells by sorting.

During the culturing period, FACS analysis was conducted several times to confirm the differentiation stages. A considerable number of dead cells were observed over the culturing period. Dead cells were preferably eliminated by using, for example, Propidium Iodide (PI) or 7-AAD before the FACS analysis.

Day 16: (Cells were subcultured.)

The cells loosely adhered to the OP9/DLL1 cells were gently dissociated by pipetting several times. The cells were passed through a 100 μm mesh and collected in a 50 conical tube. The tube was centrifuged at 1200 rpm for 7 minutes at 4° C. The pellet was dispersed in 10 mL of medium B. Thus prepared cells were seeded on the OP9/DLL1 cells in a new dish.

Day 23: (Cells were subcultured) Blood cell colonies began to appear.

The cells loosely adhered to the OP9/DLL1 cells were gently dissociated by pipetting several times The cells were passed through a 100 μm mesh and collected in a 50 mL conical tube. The tube was centrifuged at 1200 rpm for 7 minutes at 4° C. The pellet was dispersed in 10 mL of medium B.

Day 36: Stimulation of the CD4+CD8+ DP cells

In order to differentiate DP cells into CD8 SP cells, DP cells were isolated with CD4 micro beads, and the isolated cells were stimulated with medium B supplemented with anti CD3 antibody (500 ng/μL) and IL-2 (100 U/mL).

Day 43: Confirmation of CD8 positive cells

The cells were analyzed by means of FACS and the generation of CD8 single positive cells (CD8SP) was confirmed.
3) Introduction of gene encoding an HLA-C2 ligand molecule into the T-iPS cells induced from the donor homo-A

Gene encoding an HLA-C2 ligand molecule, HLA-C*04:01:01 was introduced into the T-iPS cells induced from homo-A using a Lentiviral vector. The gene was incorporated in plasmid vector CS-UbC-RfA-IRES-Venus that was obtained from Riken BioResearch Center.

The plasmid vector was introduced into the Lenti-X 293T cells by lipofection. The culture supernatant of the cells was used as lentiviral vector. iPS cells were collected by using 0.5×TrypLE select and 5×10⁴ iPS cells were dispersed in 1 mL of the supernatant containing the lentiviral vector. The lentiviral vector was infected to the iPS cells by means of spin infection (800 g, 1.5 hours, at 32° C.). The infected iPS cells were cultured and single cell colony was isolated. The introduction of the gene was confirmed by the expression of fluorescent protein, Venus.

The iPS cells were differentiated into CD8 single positive cells (homo-A CD8SP+ C*04:01:01) by the procedures shown in the above explained step 2).
4) Fractionating the NK cells

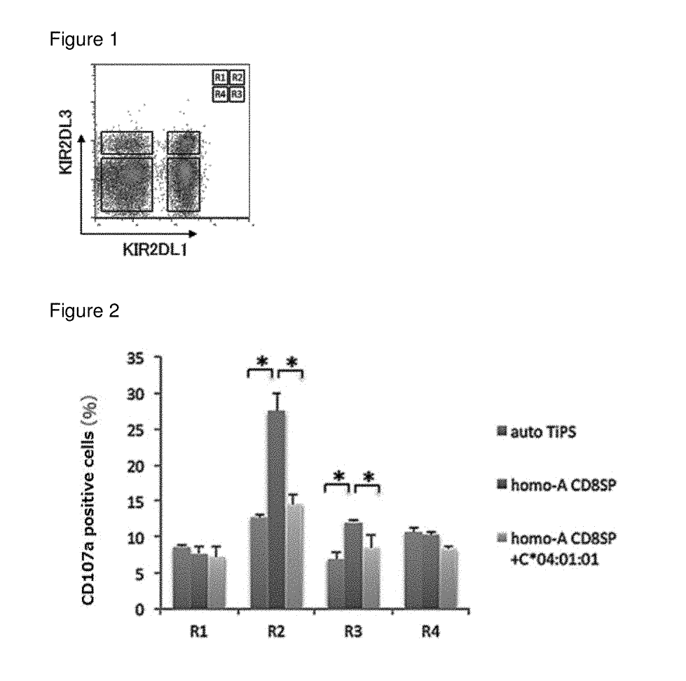

NK cells were obtained from the donor hetero-1 by the conventional procedure. The NK cells were fractionated by FACS using an antibody against KIR 2DL3, an inhibitory receptor specific for the HLA-C1 ligands, and an antibody against KIR 2DL1, an inhibitory receptor specific for the HLA-C2 ligands. As shown in FIG. 1, the cells were divided into the four fractions R1-R4.
5) NK cell activation in response to the regenerated T cells

The killer activity of the NK cells of hetero-1 against the T cells (homo-A CD8SP) that were re-generated from T-iPS cells induced from homo-A, T cells (auto T-iPS) that were regenerated T-iPS cells induced from hetero-1, and T cells (homo-A CD8SP+C*04:01:01) regenerated from T-iPS cells induced from homo-A and introduced with a gene encoding HLA-C2 ligand molecule into the genome were examined. The respective target cells and NK cells were mixed to dive effector/target cell ratio of 1:1 and incubated. After 12 hour's incubation, the expression of CD107a on the NK cell fractions was detected by FACS. The increases of CD107a on the NK cell fractions R1-R4 were analyzed. In the NK cells of fractions R2 and R3, the expressions of CD107a against CD8SP cells derived from, homo-A iPS cells were significantly increased in relation to the expression against the CD8SP cells derived from auto-iPS cells It had confirmed that the NK cells were activated in response to the home-A CD8SP cells.

On the other hand, the T cells (homo-A CD8SP-C*04:01:01) regenerated from iPS cells induced from the cells of homo-A and introduced with a HLA-C2 ligand molecule, HLA-C*04:01:01 by means of Lentiviral vector did not activated the NK cells. That is, the activation of the NK cells induced by the T cells regenerated from homo-A iPS cells having no HLA-C2 ligand molecule was significantly suppressed by the introduction of the HLA-C2 ligand molecule According to those results, T cells regenerated from cells of a donor who is homozygous for haplotype activate immune reaction of the NK cells in the recipient who is heterozygous for the HLA haplotypes and has HLA-C1/C2 ligand molecules. In addition, the activation of the NK cells in the recipient could be duly suppressed by expressing the recipient's HLA-C2 ligand molecule in the regenerated T cells. Results are shown in FIG. 2.
6) Killer activity of the NK cells against the target cells.

The regenerated T cells of homo-A CD8SP, auto T-iPS and homo-A CD8SP+C*04:01:01 were used as target cells. The NK cells and the regenerated cells were mixed to give the effector/target ratios of 2:1 and 8:1, and the mixture was incubated for 6 hours. The ratio of Annexin V positive cells was determined to confirm percentage of dead cells among the target cells. The specific lysis was calculated as follows:

Specific Lysis(%)=(% sample lysis with effector−% basal lysis without effector)/(100−% basal lysis without effector)×100

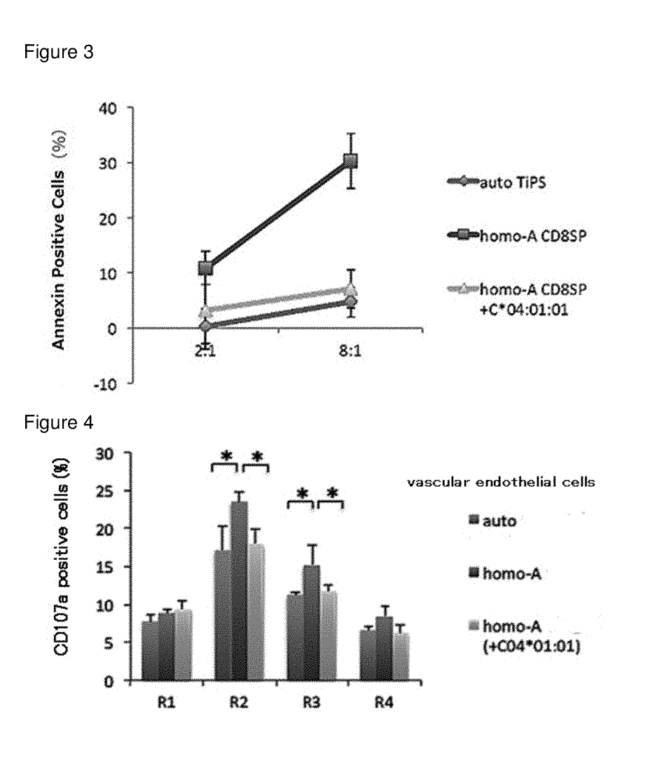

Results are shown in FIG. 3.

NK cells of hetero-1 killed the T cells regenerated from iPS cells induced from the cells of homo-A. Whereas the killer activity of the NK cells of hetero-1 against the T cells (homo-A CD8SP+C*04:01:01) regenerated from iPS cells induced from the cells of homo-A and introduced with a gene encoding an HLA-C2 ligand molecule, HLA-C*04:01:01 was significantly suppressed. According to those results, T cells regenerated from cells of a donor who is homozygous for HLA haplotype activate immune reaction of the NK cells in the recipient who is heterozygous for HLA haplotypes and has HLA-C1/C2 ligand molecules. In addition, the activation of the NK cells in the recipient can be suppressed by expressing the gene encoding the recipient's HLA-C2 ligand molecule in the regenerated T cells.

### EXAMPLE 2

1) Differentiation of iPS cells into vascular endothelial cells

iPS cells induced from the donor homo-A having homozygous HLA haplotype shown in Table 1 and iPS cells induced from the donor hetero-1 having heterozygous HLA haplotypes shown in Table 1 were prepared. iPS cells induced from the donor homo-A and introduced with HLA-C*04:01:01 into their genome were also prepared. Those iPS cells were differentiated into vascular endothelial cells.

Medium used in this example is shown below:

TABLE 4
Medium for Differentiation

| | Amount |
|---|---|
| RPMI | 485 mL |
| 200 mM L-Glutamine | 5 mL |
| B-27 Supplement Minus Insulin | 10 mL |
| Total | 50 mL |

Day 0

iPS cells were collected by using 0.5×TrypLE select and seeded on each well of a 6-well plate coated with Laminin 511 to give 2×10⁵ cells/well in the StemFit medium. The cells were incubated for 4 days until the cell culture become 100% confluent.

Day 4

The medium was replaced with 5 mL of fresh StemFit supplemented with b-FGF (4 ng/mL) and matrigel (1/60 dilution),

Day 5

The medium was replaced with 5 mL of the medium for differentiation supplemented with 10 ng/mL BMP4, 10 ng/mL b-FGF and matrigel 1/60.

Day 8, 10 and 11

The medium was replaced with 5 mL of the medium for differentiation supplemented with 100 ng/mL VEGF.

Day 13 (Collection of the cells)

The cell culture was washed with 5mL of PBS, added with 1 mL of Accumax and then, incubated for 15 minutes at 37° C. The cells were collected and dispersed in 500 μL of PBS supplemented with 5 mM EDTA and 5% FBS. 0.5 μL/10⁶ cells of α-CD31 Abs and α-VE-Cadherin Abs were added to the cell suspension and incubated at RT for 30 minutes. The cells were then washed with 10 mL of PBS supplemented with 5 mM EDTA and 5% FBS. The CD31⁺VE-Cadherin⁺ cells ere sorted using FACS Aria. The obtained vascular endothelial cells or the re-generated vascular endothelial cells were stored in a freezer at −80° C. until use.
2) NY cell activation against the re-generated vascular endothelial cells

Whether the NK cells of hetero-1 were activated against the vascular endothelial cells regenerated from iPS cells induced from the cells of a donor who is homozygous for HLA haplotypes was examined according to the procedures of Example 1. Results are shown in FIG. 4.

The regenerated vascular endothelial cells and NK cells of hetero-1 were mixed to give the effector/target ratio of 1:1 and incubated for 12 hours according to the procedures of Example 1. The expression of CD107a on the NK cells after 12 hour' a incubation was examined. NK cells of hetero-1 in R2 and R3 fractions were significantly activated by the vascular endothelial cells induced from homo-A. Those results support that not only re-generated T cells but also various re-generated cells or tissues homozygous for HLA haplotypes, i.e. having HLA-C1/C1 or HLA-C2/C2 ligand molecules activate the NK cells having both HLA-C1 and HLA-C2 ligand molecules. In addition, when vascular endothelial cells (homo-A vascular endothelial cells+C*04:01:01) regenerated from iPS cells induced from donor homo-A and introduced with HLA-C*04:01:01 into their genome were used, the activation of the NK cells of hetero-1 was significantly suppressed. This result supports that the introduction of HLA-C2 ligand molecule is also useful for the suppression of the NK cell activation.

### EXAMPLE 3

We examined whether the phenomenon that haplotype hetero NK cells having both HLA-C1/C2 ligand molecules react with regenerated cells from the cells homozygous for HLA haplotypes having C1 ligand molecule alone is a universal phenomenon. As target cells, iPS cells of strain 454E2 induced from a donor homo-B having an HLA haplotype that is most frequent in Japan in homozygous were used. iPS cell strain 454E2 was obtained from Riken. NK cells of another donor hetero-2 who was heterozygous for HLA haplotypes, one of his HLA haplotypes matches the HLA haplotype or homo-B and having HLA-C1/C2 ligand molecules were used. HLA-C2 ligand molecule, HLA-C*15:02:01 was introduced into the iPS cells induced from the cells of homo-B in the same procedure as in Example 1. The introduced gene was obtained from Riken.

TABLE 5

| | HLA-A | HLA-B | HLA-C | HLA-DRB1 |
|---|---|---|---|---|
| homo-B | 24:02 | 52:01 | 12:02 | 15:02 |
| | 24:02 | 52:01 | 12:02 | 15:02 |
| hetero-2 | 02:06 | 40:01 | 15:01 | 08:02 |
| | 24:02 | 52:01 | 12:02 | 15:02 |

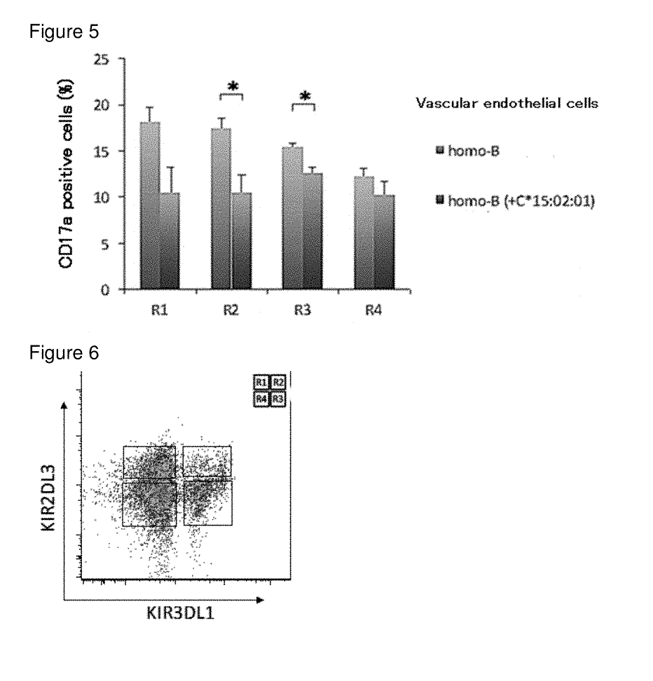

Vascular endothelial cells were regenerated from the homo-B iPS, cells and the NK cell activation test with the regenerated cells was conducted in the same manner as Example 1. Results are shown in FIG. 5. The vascular endothelial cells differentiated from homo-B iPS cells activated the NK cells of hetero-2. In contrast, vascular endothelial cells induced from homo-B iPS cells incorporated with the HLA-C2 ligand molecule of HLA-C*15:02:01 substantially suppressed the activation of the NK cells. Those results support that the introduction of gene encoding an HLA-C2 ligand molecule in HLA haplotype homo iPS cells is useful.

### EXAMPLE 4

NK cells were isolated from a heal thy volunteer Donor-NK1 by the conventional method. Peripheral blood mononuclear cells were isolated from healthy volunteers of Donor-NK1. Donor-A and Donor-B. The HLA haplotypes of the donors are shown in Table 6.

TABLE 6

| | HLA-A | HLA-B | HLA-C | HLA-DRB1 |
|---|---|---|---|---|
| Donor-NK1 | 33:03 | 44:03 | 14:03 | 13:02 |
| | 33:03 | 44:03 | 14:03 | 13:02 |
| Donor-A | 02:06 | 40:02 | 03:04 | 08:02 |
| | 11:01 | 40:02 | 03:04 | 09:01 |
| Donor-B | 02:10 | 07:02 | 07:02 | 04:05 |
| | 24:02 | 40:06 | 08:01 | 13:02 |

The HLA-C molecules of the donors used herein are HLA-C1 ligand molecules and there is no mismatch regarding the HLA-C ligands among the donors. HLA-B of Donor-NK1 is a Bw4 ligand molecule. HLA-B4403 could transmit a strong signal as a ligand to the inhibitory receptor expressed an the NK cells. HLA-B molecules of Donor-A and Donor-B are not Bw4 type ligands. HLA-A-2402 in Donor-B has been known as a weakly positive Bw4

NK cells isolated from Donor-NK1 were fractionated by FACS with antibodies against KIR3DL1 that is a HLA-Bw4 type ligand specific receptor and an antibody against KIR2DL3 that is a HLA-C1 type ligand specific inhibitory receptor. As shown in FIG. 6, the cells were divided into four fractions R1-R4.

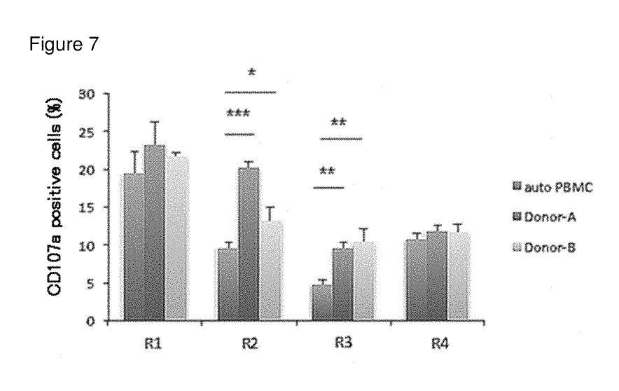

NK cell activation test was conducted using peripheral blood mononuclear cells (PBMC) isolated from Donor-NK1, Donor-A and Donor-B as target cells. The respective target cells (PBMCs) and the NK cell s were mixed to give effector/target cell ratio of 1:1 under the presence of IL-2 (1000U/mL) and incubated for 6 hours. After the incubation, the expression of CD107a on the NK cell fractions was detected by FACS. The increases of CD107a on the NK cell in the respective fractions R1-R4 were analyzed. When the expressions of CD107a increased in relation to the expression in the presence of the PBMC (auto) isolated from the Donor-NK1, the NK cells were activated. Results are shown in FIG. 7.

Whether the NK cells isolated from Donor-NK1 who had a strong Bw4 ligand molecule was reactive to the PBMC of Donor-A who did not have Bw4 was examined. “HLA-B4403” could transmit a strong signal as a ligand to the inhibitory receptor expressed in the NK cells.

In the fractions of R2 and R3, significant increases of CD107 in response to PBMC of Donor-A compared with the expression in response to the auto PMBC (PBMC of the Donor-NK1) were observed. This result support that the transplantation of tissues or cells that are Bw4 ligand negative into Bw4 ligand positive recipient could cause rejection reaction.

Next, whether the NK cells isolated from Donor-NK1 who had strong Bw4 ligand molecule were activated by PBMC of Donor-B who had HLA-A2402, a relatively weak Bw4 positive ligand was examined. As was in the case Donor-A, significant increases of the CD107a expression were observed in the R2 and R3 fractions of the NK cells co-cultured with the PBMCs derived from Donor-B. This result show that the regenerated tissues or cells that express an HLA-Bw4 ligand molecule could activate NK cells in a recipient who has a strong HLA-Bw4 ligand molecule when the HLA-Bw4 ligand molecule expressed in the cells or tissues is a weakly positive ligand.

What is claimed is:

1. A method for preparing cultured cells or tissues for transplantation, comprising at least one of the following steps 1) and 2):
1) when the cultured cells or tissues do not express an HLA-C molecule of at least one HLA-C groups expressed in the receipient's HLA-C locus, forcing the expression of the HLA-C molecule of said HLA-C group in the cultured cells or tissues, or
2) when the cultured cells or tissues are negative or weakly positive for HLA-Bw4 while the recipient is positive for HLA-Bw4, forcing the expression of an HLA molecule of HLA-Bw4 group in the cultured cells or tissues.

2. The method according to claim 1, wherein the cultured cells or tissues for tranplantation are those induced from stem cells or progenitor cells.

3. The method according to claim 2, wherein the cultured cells or tissues for tranplantation are differentiated in vitro from ES cells or iPS cells.

4. The method according to claim 3, wherein the the cultured cells or tissues for tranplantation are those differentiated from iPS cells induced from a cell of a donor who is homozygous for HLA haplotypes.

5. The method according to claim 4, wherein the donor who is homozygous for HLA haplotypes is homozygous for at least HLA-A, HLA-B and HLA-DR.

6. The method according to claim 5, wherein the donor is homozygous for HLA-C.

7. The method according to claim 5, wherein the iPS cells induced from a cell of a donor homozygous for HLA haplotypes are obtained from an iPS cell bank in which iPS cells induced from donors who are homozygous for HLA haplotypes are stored in connection with information regarding HLA of the donors.

8. The method according to claim 4, wherein the step of forcing the expression of the HLA-C ligand molecule or HLA-Bw4 ligand molecule in the cultured cells or tissues for transplantation in the steps 1) or 2) comprises the steps of:
introducing a gene encoding the desired HLA-C ligand molecule and/or HLA-Bw4 ligand molecule into the iPS cells, and differentiating the iPS cells into the desired cells or tissues to be transplanted.

9. The method according to claim 1, wherein the step of forcing the expression of the HLA-C ligand molecule or HLA-Bw4 ligand molecule in the cultured cells or tissues for transplantation in the step 1) or 2) comprises the step of:
introducing a gene encoding the desired HLA-C ligand molecule and/or HLA-Bw4 ligand molecule into the desired cells or tissues to be transplanted.

10. The method according to claim 1, wherein the HLA-C ligand molecule or HLA-Bw4 ligand molecule to be introduced into the cultured cells or tissues for transplantation in the step 1) or 2) is the same HLA-C ligand molecule or HLA-Bw4 ligand molecule as that expressed in the recipient.

11. iPS cells that are induced from a cell of a donor who is homozygous for at least HLA-A, HLA-B and HLA-DR, and having at least one additional mole that is not derived from the donor from whom the iPS cells were induced, and the additional HLA molecule is selected from the group of (1) and (2):
(1) an HLA molecule of HLA-C1 and/or HLA-C2 group, or
(2) an HLA molecule of HLA-Bw4 group.

12. An iPS cell bank, comprising the iPS cells of claim 11 that are stored in connection with information regarding donor's HLA and HLA molecules introduced into the cells.

13. A method for creating an iPS cell bank for transplantation, which comprising the steps of:
(1) preparing iPS cells induced from a donor who is homozygous for at least HLA-A, HLA-B and HLA-DR,
(2-1) when the donor has HLA-C1/C1 ligand molecules at the HLA-C locus, introducing a gene encording an HLA-C2 ligand molecule into the iPS cells; when the donor has HLA-C2/C2 ligand molecules at the HLA-C locus, introducing a gene encording an HLA-C1 ligand molecule into the iPS cells, and/or
(2-2) when the donor is negative or weakly positive for HLA-Bw4, introducing a gene encoding an HLA-Bw4 ligand molecule into the iPS cells, and
(3) storing the iPS cells obtained in step (2-1) and/or (2-2) in connection with information regarding HLA of the donor and the HLA molecule introduced into the iPS cells.
